(19) United States
(12) Patent Application Publication
(10) Pub. No.: US 2022/0257099 A1
(43) Pub. Date: Aug. 18, 2022

(54) FLUID MANAGEMENT SYSTEM

(71) Applicant: Keymed (Medical & Industrial Equipment) Limited, Southend-on-Sea (GB)

(72) Inventor: Daniel TRIGG, Southend-on-Sea (GB)

(73) Assignee: Keymed (Medical & Industrial Equipment) Limited, Southend-on-Sea (GB)

(21) Appl. No.: 17/571,855

(22) Filed: Jan. 10, 2022

(30) Foreign Application Priority Data
Feb. 17, 2021 (GB) 2102202.5

(51) Int. Cl. A61B 1/015; G16H 40/63; A61B 1/00

(52) U.S. Cl. CPC A61B 1/015; A61B 1/0004; A61B 1/00006; G16H 40/63

(57) ABSTRACT

A fluid management system for an endoscopic procedure including: a pump configured to deliver liquid from a reservoir to a medical device; a processor comprising hardware, the processor being configured to control operation of the pump; a footswitch in communication with the processor, the footswitch being configured to actuate the pump; and a user input device configured to program the processor to operate the pump at a plurality of predetermined levels of an operational parameter of the pump and to allow a user to select one of the plurality of predetermined levels for pump operation, wherein the processor is further configured to disable at least parts of the user input device when the footswitch is engaged.

### CROSS-REFERENCE TO RELATED APPLICATION

The present application is based upon and claims the benefit of priority from GB 2102202.5 filed on Feb. 17, 2021, the entire contents of which is incorporated herein by reference.

### BACKGROUND

### Field

The present disclosure relates to a fluid management system for medical procedures, such as endoscopies.

### Prior Art

Endoscopic procedures involve the insertion of a medical instrument into a patient's body to observe an internal organ or tissue in detail for the purposes of imaging, investigation, diagnosis or surgery/treatment. Such endoscopic procedures often involve supplying irrigation fluid into the patient's body to irrigate and clean the site being observed. For example, in gastrointestinal (GI) procedures irrigation is helpful to wash gastric and colonic mucosa and to remove blood, faeces or other organic matter. Irrigation fluid is supplied by a pump into a channel in the endoscope itself. The surgeon carrying out the procedure may operate the pump by means of a footswitch.

### SUMMARY

Accordingly, a fluid management system for an endoscopic procedure is provided. The fluid management system comprising a pump configured to deliver liquid from a reservoir to a medical device, the pump comprising a central processing unit (CPU) configured to control operation of the pump, a footswitch in communication with the CPU configured to actuate the pump, and a user input device configured to program the CPU to operate the pump at a plurality of predetermined levels of an operational parameter of the pump and to allow a user to select one of the plurality of predetermined levels for pump operation, wherein the CPU is further configured to disable at least parts of the user input device when the footswitch is engaged.

The fluid management system can prevent sudden changes in an operational parameter of the pump during use, such as the flow rate of irrigation fluid, in order to prevent potential harm to the patient or damage to the equipment.

The user input device can comprise increment controls operable to incrementally increase and decrease an operational parameter of the pump, and preset controls operable to switch directly between the plurality of predetermined levels of the operational parameter of the pump.

The CPU can be configured to disable the preset controls when the footswitch is engaged and to maintain the increment controls in an enabled condition when the footswitch is engaged.

The user input device can comprise a plurality of buttons on the pump body which are illuminated when enabled.

The pump can comprise a peristaltic pump and the operational parameter can be at least one of pump speed or flowrate.

### BRIEF DESCRIPTION OF THE DRAWINGS

The embodiments will now be described in detail, by way of example only, with reference to the accompanying drawing in which:

FIG. 1 illustrates a schematic view of a fluid management system; and

FIG. 2 illustrates an enlarged view of the user input device.

### DETAILED DESCRIPTION

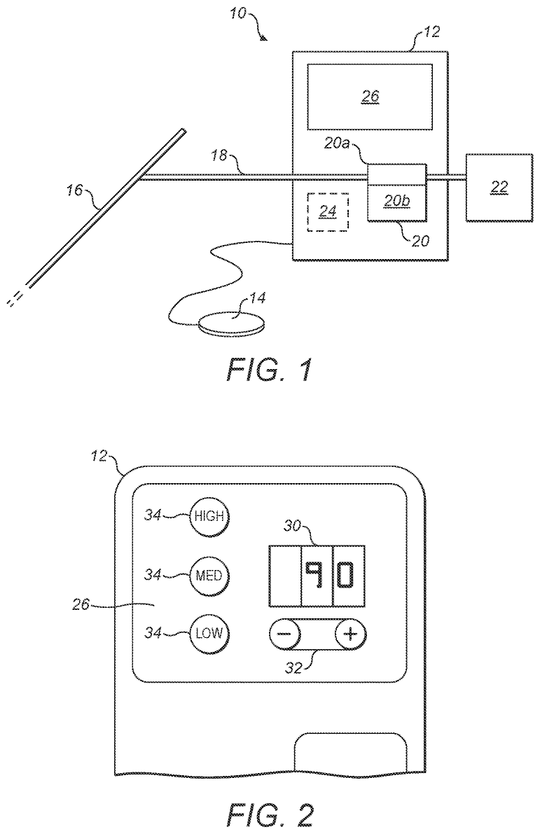

As shown schematically in FIG. 1, a fluid management system 10 comprises a pump 12 and a footswitch 14 connected to the pump 12. When the footswitch 14 is engaged, the pump 12 supplies a medical instrument 16, such as an endoscope, with fluid via tubing 18.

The pump 12 can be a peristaltic pump which houses a drive or motor (not shown) for operating a pump head 20. The pump head 20 includes a track 20a and rotor 20b. Flexible tubing 18 is fitted between the track 20a and rotor 20b and in operation fluid is withdrawn from a reservoir 22 and pumped through the tubing 18. The pump 12 also includes a processor, such as a central processing unit (CPU) 24, which controls operation of the pump 12. A user input device 26 is also provided. The user input device 26 can be in the form of a control panel on the body of the pump 12, although it could also be any type of device that accepts a user input, such as a touchscreen, keyboard, joystick, and may also be a remote device in wired or wireless communication with the pump 12. The CPU 24 is programmable by the user input device 26 to alter certain parameters of operation of the pump 12.

The user input device 26 can include a number of controls operable by a user to change one or more parameters of pump operation, and a display 30, as shown in FIG. 2. The adjustable parameters of pump operation may be, for example, the motor speed, the rotations per minute (RPM) of the rotor 20b, or the flow rate of fluid in the tubing 18. The display 30 may indicate an actual value of an operational parameter, or a percentage value. For example, in FIG. 2 the display 30 shows a value of 90 which may represent 90% of the maximum available flow rate which the pump 12 can provide.

The footswitch 14 is connected to the pump 12 and is in communication with the CPU 24. When the footswitch 14 is pressed by a user, the CPU 24 will cause the pump 12 to operate. When the footswitch 14 is disengaged, the CPU 24 ceases operation of the pump 12.

The controls of the user input device 26 can include increment controls 32 which allow a given parameter to be increased or decreased in successive small increments. The increment controls 32 may consist of two buttons marked+ and—as illustrated in FIG. 2, to increase and decrease the parameter, although other forms of control could be used, such as a rotatable knob or wheel, or a slider. The increment controls 32 may be configured to alter a parameter by any desired increment, such as 5% or 10% or some other value.

The controls of the user input device 26 can also include preset controls 34 which allow a user to set, and shift between, a plurality of predetermined levels, or presets, of a particular parameter. The pump 12 can be programmed to operate with a given parameter at certain levels, such as at certain percentages of the maximum available level which can be provided by a given pump. For example, a user may wish to use different fluid flow rates for different procedures or at different times during a procedure. To facilitate these changes and to avoid the need to repeatedly operate the increment controls 32 to increase or decrease the flow rate (or other chosen parameter) prior to a procedure being carried out, presets may be entered corresponding to the desired flow rates. For example, the presets may be a maximum flow rate of 90% of the pump's operational capacity, a medium flow rate of 50% and a minimum flow rate of 10% may be set.

The preset controls 34 can comprise three buttons representing maximum, medium and minimum levels. The preset controls 34 may be labelled accordingly, or labelled High, Med and Low, as in FIG. 2, or identified in any other convenient manner More or fewer buttons may be provided depending on the number of presets required, and other forms of control such as switches could be used. The chosen parameter may be set initially by using the increment controls 32. When the desired level is reached, as shown on the display 30, the corresponding preset button may be held for a several seconds to set that level as the desired preset. For example, if the maximum required is 90% as shown in FIG. 2, the High button may be held for several seconds to fix that value as the high preset. The High button is then released, the level of the chosen parameter lowered to a required medium value using the increment controls 32, and that level is fixed by holding the Medium button for several seconds and so on. Once the preset controls 34 are all programmed in this way, pressing one of the buttons automatically changes the parameter to the given preset level.

In use, the pump 12 is programmed by a user, using the user input device 26, with the desired parameter levels. These levels may be based on recommended levels for given procedures or may be chosen by an individual user based on their knowledge and experience. During a procedure, the user chooses a desired level by pressing the relevant preset control 34 and then engages the footswitch 14 in order to start and stop pump operation. To change the parameter, e.g., the flow rate, the user presses one of the buttons of the user input device 26. Small changes may be made by using the increment controls 32 and larger changes by choosing a different one of the preset controls 34.

However, if the wrong preset control 34 is pressed inadvertently, e.g. if a change from minimum to maximum is chosen by mistake when a change from minimum to medium was intended, or if the parameter levels which have been programmed as the relevant presets have in fact been altered prior to the procedure being carried out and are not what the user is expecting, there is a risk of a sudden increase or decrease in the parameter, such as flow rate, which could cause injury to the patient, damage to the endoscopic equipment, or otherwise interfere with the efficient carrying out of the procedure.

Therefore, the system can be configured to prevent any alteration to the presets, or switching between presets, while the pump 12 is in operation. The user input device 26, at least in so far as it relates to the preset controls 34, is disabled (e.g., an input from the preset controls to the CPU is ignored by the CPU) while the pump 12 is operating, that is, while the footswitch 14 is engaged. The relevant parts of the user input device 26 can be disabled by means of the CPU 24, which is configured to detect engagement and disengagement of the footswitch 14 and is programmed to disable the preset controls 34 when the footswitch 14 is engaged so that step changes from one preset level to another, or changes to level of each preset, are not permitted.

However, the increment controls 32 may not be disabled so that the parameter can still be altered during operation of the pump 12, but only small incremental changes are possible. Therefore, the parameter, such as the flow rate, can be changed but only gradually in a way which will not cause injury or damage.

The control buttons on the user input device 26 may be illuminated when they are enabled. The CPU 24 may also be operable to stop illumination of the disabled preset control 34 when the footswitch 14 is engaged, to make the user aware that they are inoperable. The increment controls 32 can remain illuminated even when the footswitch 14 is engaged, to ensure a user is aware they are still operable and the flow rate changes can be made incrementally.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. A fluid management system for an endoscopic procedure, the fluid management system comprising:
a pump configured to deliver liquid from a reservoir to a medical device;
a processor comprising hardware, the processor being configured to control operation of the pump;
a footswitch in communication with the processor, the footswitch being configured to actuate the pump; and
a user input device configured to program the processor to operate the pump at a plurality of predetermined levels of an operational parameter of the pump and to allow a user to select one of the plurality of predetermined levels for pump operation;
wherein the processor is further configured to disable at least parts of the user input device when the footswitch is engaged.

2. The fluid management system as claimed in claim 1, wherein the user input device comprises increment controls operable to incrementally increase and decrease the operational parameter of the pump, and preset controls operable to switch directly between the plurality of predetermined levels of the operational parameter of the pump.

3. The fluid management system as claimed in claim 2, wherein the processor is configured to disable the preset controls when the footswitch is engaged and to maintain the increment controls in an enabled condition when the footswitch is engaged.

4. The fluid management system as claimed in claim 3, wherein the user input device comprises a plurality of control buttons on a pump body of the pump which are illuminated when enabled.

5. The fluid management system as claimed in claim 1, wherein the pump comprises a peristaltic pump.

6. The fluid management system as claimed in claim 5, wherein the operational parameter is at least one of pump speed or flow rate of liquid being pumped.

7. The fluid management system as claimed in claim 1, further comprising a pump unit, the pump unit comprising the pump, the processor and the user input device.

8. A control system comprising a processor configured to:
control operation of a pump configured to deliver liquid from a reservoir to a medical device;
detect whether a footswitch is configured to actuate the pump;
operate the pump based on a first input from a user input device indicating a plurality of predetermined levels of an operational parameter of the pump; and
ignore the first input from the user input device when the footswitch is engaged.

9. The control system as claimed in claim 8, wherein:
the first input indicates a plurality of predetermined levels of the operational parameter of the pump; and
the processor is further configured to receive a second input from the user input device indicating an incremental increase and decrease of the operational parameter of the pump.

10. The control system as claimed in claim 9, wherein the processor is configured to ignore the first input while the footswitch is engaged and to control the pump to incrementally increase and decrease the operational parameter of the pump when the second input is received while the footswitch is engaged.

11. The control system as claimed in claim 10, wherein the user input device comprises a plurality of control buttons, the processor being configured to control an illumination of one or more of the plurality of control buttons when the one or more of the plurality of control buttons are enabled.

12. The control system as claimed in claim 8, wherein the operational parameter is at least one of pump speed or flow rate of liquid being pumped.

13. A method for controlling operation of a pump configured to deliver liquid from a reservoir to a medical device, the method comprising:
detecting whether a footswitch is configured to actuate the pump;
operating the pump based on a first input from a user input device indicating a plurality of predetermined levels of an operational parameter of the pump; and
ignoring the first input from the user input device when the footswitch is engaged.

14. The method as claimed in claim 13, wherein:
the first input indicates a plurality of predetermined levels of the operational parameter of the pump; and
the method further comprises receiving a second input from the user input device indicating an incremental increase and decrease of the operational parameter of the pump.

15. The method as claimed in claim 14, wherein the ignoring comprises ignoring the first input while the footswitch is engaged and controlling the pump to incrementally increase and decrease the operational parameter of the pump when the second input is received while the footswitch is engaged.

16. The method as claimed in claim 15, wherein the user input device comprises a plurality of control buttons, and the method further comprises controlling an illumination of one or more of the plurality of control buttons when the one or more of the plurality of control buttons are enabled.

17. The method as claimed in claim 13, wherein the operational parameter is at least one of pump speed or flow rate of liquid being pumped.
